(19) United States
(12) Patent Application Publication
(10) Pub. No.: US 2022/0137042 A1
(43) Pub. Date: May 5, 2022

(54) Lateral Flow Assay Device And Sampling Methods

(71) Applicant: Signature Science, LLC, Austin, TX (US)

(72) Inventors: Stephen Straight, Austin, TX (US); J. KIRBY KENDALL, Cedar Park, TX (US); WILLIAM TWEDDELL, Austin, TX (US)

(21) Appl. No.: 17/519,054

(22) Filed: Nov. 4, 2021

Related U.S. Application Data
(60) Provisional application No. 63/109,551, filed on Nov. 4, 2020.

(51) Int. Cl. G01N 33/543; B01L 3/00

(52) U.S. Cl. CPC G01N 33/54388; B01L 3/5023; B01L 2300/069; B01L 2400/0683; B01L 2300/0672; B01L 3/523

(57) ABSTRACT

Disclosed is a lateral flow assay device useful for useful for detecting a variety of analytes in samples. Also disclosed are methods of using the device and other lateral flow assay sampling and detection methods. In general, the devices and methods allow for a lateral flow assay test to be conducted within a single, closed environment, thus minimizing risks of sample contamination as well as other potential hazards to the user.

### CROSS-REFERENCE TO RELATED APPLICATIONS

This application claims priority to U.S. Provisional Application No. 63/109,551, filed Nov. 4, 2020, the entirety of which is hereby incorporated by reference herein.

### FIELD

This application relates generally to a lateral flow assay device useful for detecting a variety of analytes in samples. The application also relates to methods of using the device and other lateral flow assay sampling and detection methods.

### BACKGROUND

To detect an analyte using a lateral flow assay, a liquid or dissolved sample must flow along a media until the colorimetric region of the assay is reached. In general, existing lateral flow assays are either prepackaged assays that include the needed reagents, or inexpensive lateral flow assays known as “test strips.” Use of these existing products requires the user to collect the material to be analyzed, add it to a vial of buffer, shake the vial, and then pipette the liquid in the vial into a cassette that holds the immunoassay strip.

One disadvantage of such products is that contamination and risk of exposure to hazardous analytes are increased due to the fact that the test must be carried out in different environments. For example, there is a risk of contamination or hazard to a user when liquid from the buffer vial is transferred onto the immunoassay strip. Accordingly, there is a need in the art for devices and methods that reduce such risks. This need and others are met by the following disclosure.

### SUMMARY

Disclosed herein, in one aspect, is a device configured to house a lateral flow assay strip. The device can comprise a housing moveable from an open position to a closed position. When the housing is in the closed position, it can define an enclosed space comprising a receptacle configured to receive the lateral flow assay strip.

The device can further comprise a sample collection pad. At least a portion of the sample collection pad can be received within the enclosed space when the housing is in the closed position. The device can further comprise a fluid reservoir configured to allow fluid transfer from the reservoir to the sample collection pad when the housing is in the closed position.

Also disclosed herein, in a further aspect, is a method for using a disclosed device. The method can comprise contacting the sample collection pad with a sample, releasing a fluid from the fluid reservoir, thereby causing the fluid to contact the sample collection pad and diffuse onto the lateral flow assay strip, and detecting a response from the lateral flow assay strip that is indicative of the presence or absence of an analyte in the sample.

In a further aspect, disclosed herein is a method of analyzing a sample using a lateral flow assay. The method can comprise contacting the sample with a sample collection pad that is in fluid communication with a lateral flow assay strip, and causing a fluid to be released onto the sample collection pad, thereby diffusing at least a portion of the sample onto the lateral flow assay strip, followed by detecting a response from the lateral flow assay strip that is indicative of the presence or absence of an analyte in the sample. The method can be carried out in a closed environment, e.g., entirely within a closed housing that permits a user to observe the assay response without opening the closed housing.

Additional advantages of the device and methods will be set forth in part in the description which follows, and in part will be understood from the description, or may be learned by practice of the disclosed device and methods. The advantages of the disclosed device and methods will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed device and methods and together with the description, serve to explain the principles of the disclosed device and methods.

FIGS. 1A-1B depict an exemplary embodiment of a disclosed lateral flow assay device in the open (FIG. 1A) and closed (FIG. 1B) positions.

FIG. 2 is a cross-sectional view of the lateral flow assay device depicted in FIGS. 1A-B.

FIG. 3A is a transparent view of another exemplary, pen-type embodiment of a disclosed lateral flow assay device.

FIGS. 3B-3C depict the lateral flow assay device of FIG. 3A in the open (FIG. 3B) and closed (FIG. 3C) positions.

FIGS. 4A-4B depict transparent views of an alternative, pen-type embodiment of a lateral flow assay device in the open (FIG. 4A) and closed (FIG. 4B) positions.

FIG. 5 depicts an exploded view of a detailed example of the embodiment shown in FIGS. 4A-B.

### DETAILED DESCRIPTION

The disclosed device and methods may be understood more readily by reference to the following detailed description of particular embodiments and the examples included therein and to the Figures and their previous and following description.

### A. Definitions

It is to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural references unless the context clearly dictates otherwise. Thus, for example, reference to “a receptacle” includes a plurality of such receptacles, and reference to “the receptacle” is a reference to one or more receptacles and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed appliance and method belong. Although any filter conditioning unit and method similar or equivalent to those described herein can be used in the practice or testing of the present unit and method, the particularly useful units and methods are as described.

Throughout the description and claims of this specification, the word “comprise” and variations of the word, such as “comprising” and “comprises,” means “including but not limited to,” and is not intended to exclude, for example, other elements, components, integers or steps. In particular, in methods stated as comprising one or more steps or operations, it is specifically contemplated that each step comprises what is listed (unless that step includes a limiting term such as “consisting of”), meaning that each step is not intended to exclude, for example, other elements, components, integers or steps that are not listed in the step.

“Lateral flow assay strip,” as used herein, refers to an assay strip having a first end and an opposing second end, with a colorimetric region therebetween.

### B. Lateral Flow Assay Device

According to one aspect, the disclosed lateral flow assay device generally comprises a housing suitable for enclosing at least a portion of a sample collection pad and a lateral flow assay strip. A sample can be placed on the sample collection pad, for example, by opening the housing, or in some aspects, a portion of the sample collection pad can be exposed by removing a portion of the housing. Once a sample is present on the pad, a fluid reservoir can be activated (e.g., pierced) to allow release of fluid from the reservoir onto the sample collection pad.

The fluid can then diffuse from the sample collection pad to a first end of the lateral flow assay strip and proceed to an opposing second end of the lateral flow assay strip. Once the fluid reaches the colorimetric region between the first and second ends of the assay strip, the presence of the analyte can be detected. During the transport of the fluid across the lateral flow assay strip, i.e., from the first end to (or toward) the second end, the fluid or an agent within the assay can chemically and/or physically alter the analyte by forming a salt or by binding with the analyte. As the analyte (altered or not) passes the colorimetric region of the assay, the presence or absence of a color can indicate whether or not the analyte is detected. The presence or absence of the analyte can be detected, according to some embodiments, without opening the housing, e.g., by viewing the colorimetric region of the assay strip through a transparent region of the housing.

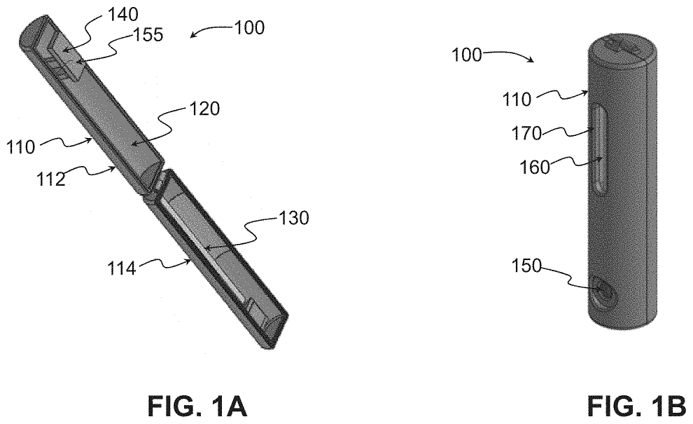

With reference to FIGS. 1A-1B, an exemplary device 100 can comprise a housing 110. Housing 110 can be moveable about and between an open position, as shown in FIG. 1A, and a closed position, as shown in FIG. 1B. At least a portion of the interior of the housing 110 can define a space 120. When the housing is in the closed position, space 120 becomes an enclosed space. In one aspect, housing 110 can comprise a first housing portion 112 and a second housing portion 114. The first and second housing portions 112, 114 can cooperate to define the space 120 (e.g., an enclosed space). In one aspect, as shown in FIGS. 1A-1B, the first and second housing portions can be hingedly coupled and configured to rotate about and between the open and closed positions of the housing. The housing 110 can be made from any suitable, non-water permeable material, such as plastic, e.g., injection molded plastic.

Housing 110 can further comprise a receptacle 130 that is configured to receive a lateral flow assay strip. Receptacle 130 can be any structure that permits a lateral flow assay strip to be secured within the enclosed space of housing 110. For example, in one aspect, receptacle 130 can comprise an adhesive for securing the assay strip onto the receptacle. Additionally, or alternatively, it is contemplated that the receptacle 130 can comprise one or more structural features, such as a brace, that physically engage and/or support a portion of the assay strip.

Device 100 can further comprise a sample collection pad 140. At least a portion of the sample collection pad 140 can be received within the space 120 when housing 110 is in the closed position. Thus, in one aspect, the housing 110 of device 100 can be moved to an open position, exposing sample collection pad 140, as shown in FIG. 1A. A sample can be collected onto sample collection pad 140, and the first and second portions 112, 114 of housing 110 can be moved to the closed position. The lateral flow assay method can then be carried out within the closed environment provided by the housing. The device can therefore minimize contamination that can result with existing lateral flow assay methods. The sample collection pad 140 can include any suitable material, such as glass fiber, open cell foam, a cellulose-based material, filter paper, or other suitable synthetic materials.

Device 100 can further comprise a fluid reservoir 150. Fluid reservoir 150 can be in fluid communication with the sample collection pad 140 when housing 110 is in the closed position. Fluid reservoir 150 and sample collection pad 140 can be arranged in any suitable configuration to permit fluid from the fluid reservoir to contact the sample collection pad. Thus, in some aspects, the fluid reservoir and sample collection pad need not be physically touching. In one aspect, fluid reservoir 150 can be a blister pack. Optionally, in this aspect, the fluid reservoir 150 can be securely received within an opening 148 that is defined within a housing portion to permit contact with the fluid reservoir from outside the housing by a user of the device. For example, the fluid reservoir 150 can comprise a flange 156 that couples to the housing 110 (e.g., via an adhesive). It is contemplated that the flange 156 and housing 110 can form a seal to inhibit fluid communication through the opening 148.

In some aspects, the fluid reservoir 150 can comprise a flexible portion 152 (e.g., comprising flexible polymer) that extends outwardly from the housing and an inflexible portion 154 (e.g., a foil) that is configured to tear when pressure within the reservoir 150 reaches a threshold, at which point the inflexible portion 154 opens to release the fluid. In some optional aspects, the inflexible portion 154 can comprise a weakened portion that is configured to break to release the fluid. Accordingly, a user can apply a force against (e.g., press) the flexible portion 152 to increase the pressure within the fluid reservoir 150 until the inflexible portion 154 breaks to release the fluid to the sample collection pack.

In a further aspect, fluid reservoir 150 can be configured such that when the first and second housing portions are moved to a closed position, fluid reservoir 150 bursts and releases fluid onto the sample collection pad. For example, it is contemplated that one of the housing portions can comprise or be coupled to a structure that is configured to pierce the fluid reservoir 150 when the housing portions are moved to the closed position. For example, the housing 110 can comprise a blade or pin point 155 that is disposed within the space 120 so that closure of the housing brings the blade or pin point into engagement with and at least partially through the fluid reservoir 150 (e.g., via piercing or puncturing) to release the fluid. In general, fluid reservoir 150 can contain a suitable fluid for lateral flow assay sampling methods, such as a buffer capable of maintaining a fluid pH of from 6 to 8.5, e.g., from 6 to 8 or from 6 to 7.4. In one aspect, fluid reservoir can contain, or be configured to receive, phosphate buffered saline and at least one surfactant.

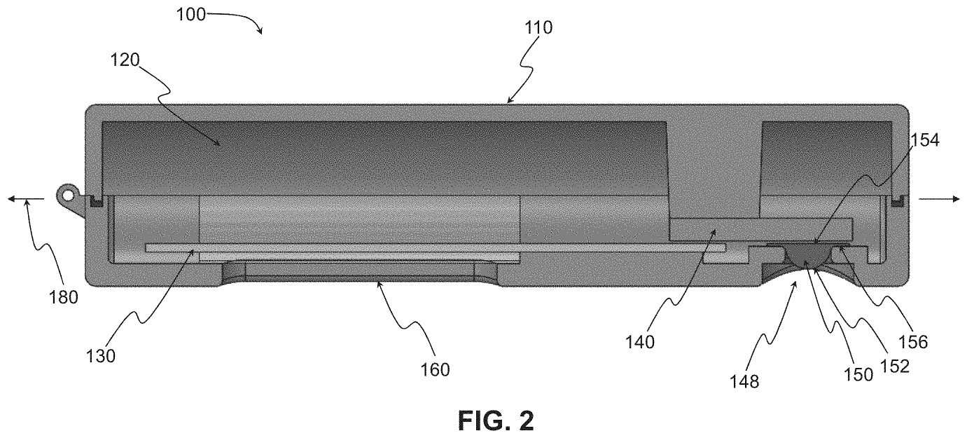

According to one aspect, housing 110 can comprise at least one transparent region 160 that is positioned relative to (e.g., overlaps with) receptacle 130 (i.e., the receptacle for the lateral flow assay strip) along an axis 180 when housing 110 is in the closed position to permit viewing of the lateral flow assay strip. In a further aspect, the at least one transparent region 160 can be defined by a window 170 within the body of housing 110. Thus, in some aspects, a response from the colorimetric region of the lateral flow assay strip can be detecting without opening the device, e.g., by viewing any response through the transparent region of housing 110. A cross-sectional view of device 100 is depicted in FIG. 2.

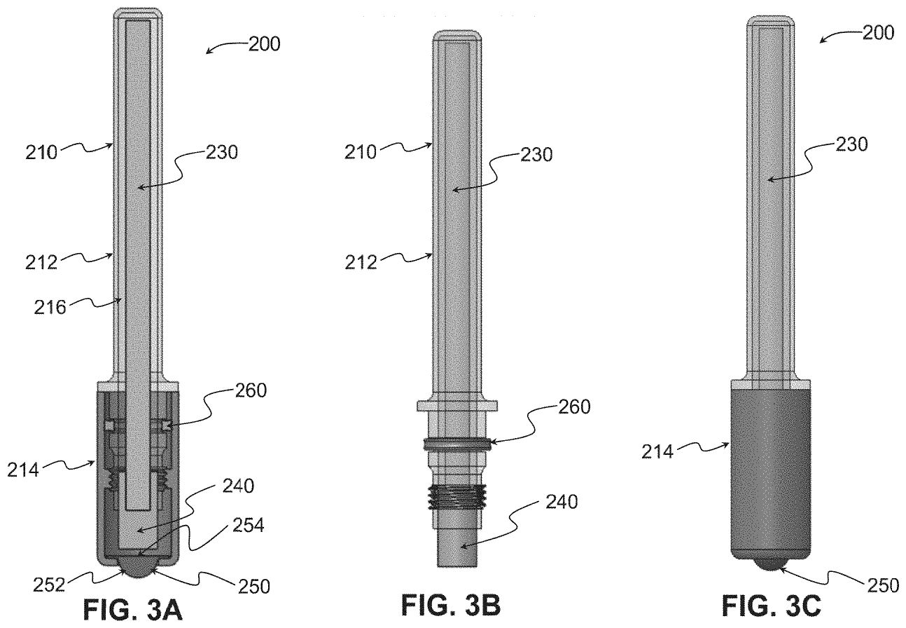

FIGS. 3A-3C depict another exemplary embodiment of a disclosed lateral flow assay device. Device 200 can comprise a housing 210. Housing 210 can include a first housing portion 212 (shown as the upper portion) and a second housing portion 214 (shown as the lower portion). First housing portion 212 can be configured to threadably couple to second housing portion 214 in a closed configuration. In the closed configuration, the housing 210 can define an enclosed space 216. At least the first portion 212 of the housing can define an enclosed space in which a receptacle 230 for a lateral flow assay strip can be located. The housing can be made from any suitable material, such as treated paperboard or plastic, e.g., injection or blow molded plastic. Device 200 can further comprise a sample collection pad 240 and a fluid reservoir 250 that can be put into fluid communication with the sample collection pad. For example, the fluid reservoir can be pierced or otherwise opened so that fluid from the fluid reservoir 250 can be communicated to the sample collection pad. Still further, in some optional aspects, the fluid reservoir, once opened, can be put in physical contact with the sample collection pad 240 to enable fluid communication therebetween. The sample collection pad can include any suitable material, such as glass fiber, open cell foam, a cellulose-based material, filter paper, wool or other suitable materials. Non-limiting examples of sample collection pad materials include linear polyester fibers and polyester wool. Optionally, the fluid reservoir 250 can be securely received within an opening at the end of the second housing portion 214 that is spaced from the first housing portion 212.

Thus, according to this aspect, device 200 can be in the open position, as is partially depicted in FIG. 3B, which can allow for sampling of a material onto sample collection pad 240. The second portion 214 of the housing can then be threadably coupled to first portion 212 (with the first and second portions having respective complementary threaded portions), such that the housing is closed and defines an enclosed space, as depicted in FIG. 3C. The device can include a fluid-resistant interface between first portion 212 and second portion 214. As depicted in FIGS. 3A-3B, for example, the device can include a sealing member 260 for sealing the first and second portions of the housing. Sealing member 260 can be an O-ring, gasket, or flange created from the material used for housing 210 or another suitable material. Although sealing member 260 is shown in FIGS. 3A-3B as being part of first portion 212, a sealing member can alternatively be present as part of section portion 214 as long as there is a fluid-resistant interface between first portion 212 and second portion 214.

Fluid reservoir 250 can be a blister pack or fluid container suitable for storing fluids without unwanted leakage. Fluid reservoir 250 (e.g., a blister pack) can then release fluid, e.g., by manual manipulation or actuation, onto sample collection pad 240. For example, the fluid reservoir 250 can comprise a flexible portion 252 (e.g., comprising flexible polymer) that extends outwardly from the housing and an inflexible portion 254 (e.g., a foil) that is configured to tear when pressure within the reservoir 250 reaches a threshold, at which point the inflexible portion 254 opens to release the fluid. In some optional aspects, the inflexible portion 254 can comprise a weakened portion that is configured to break to release the fluid. Accordingly, a user can apply a force against (e.g., press) the flexible portion 252 to increase the pressure within the fluid reservoir 250 until the inflexible portion 254 breaks to release the fluid to the sample collection pack. Thus, the fluid reservoir 250 can configured to release the fluid when subject to a user-applied pressure (e.g., a predetermined threshold pressure that is a function of a structural integrity of the fluid reservoir 250).

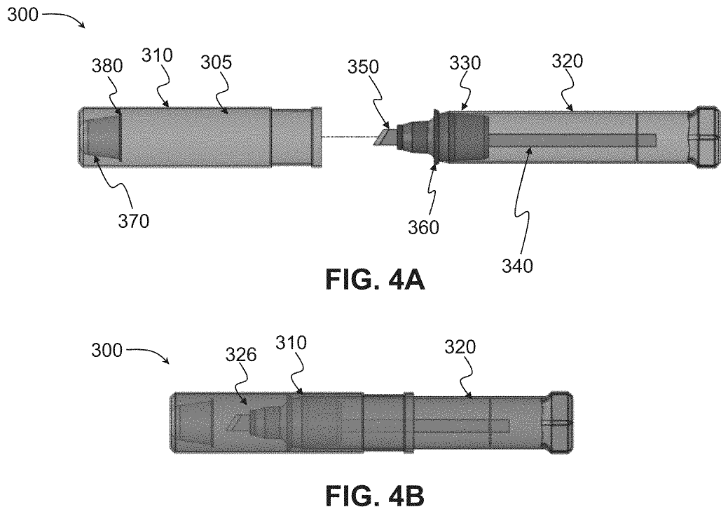

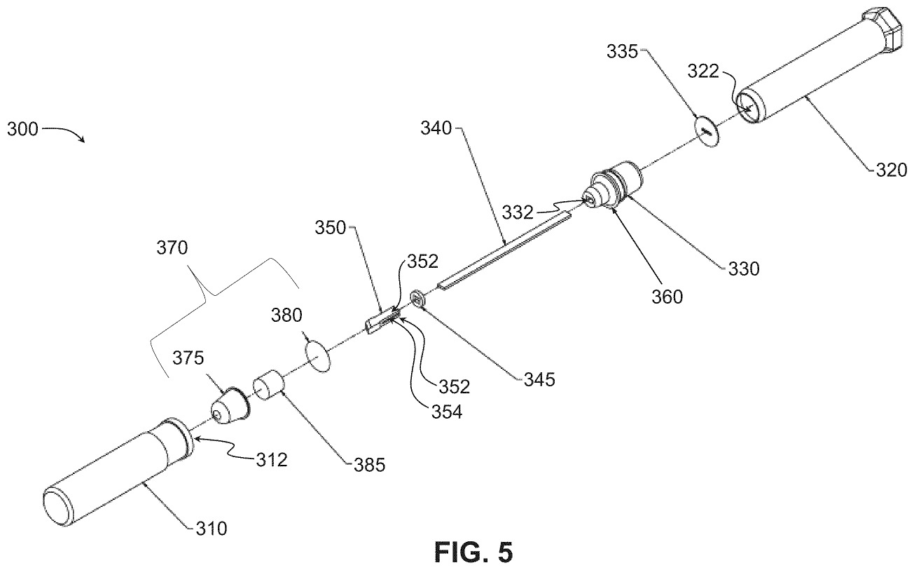

In further aspects, reservoir 250 can be sealed on one end by a pierceable foil membrane. According to this aspect, fluid can be released to sample collection pad 240 when pad 240 pierces the foil membrane and makes fluidic contact with the fluid. For example, in some optional aspects, the sample collection pad 240 can define a point or a sharp edge (as illustrated in the embodiment shown in FIGS. 4A-5). In these aspects, in some embodiments, coupling the first housing portion 212 to the second housing portion 214 (e.g., screwing the respective housing portion together) can draw the point or sharp edge at least partially through the reservoir (e.g., through an outer wall of the reservoir). In further aspects, with the first housing portion 212 and the second housing portion 214 coupled together, the reservoir 250 can remain spaced from the point or sharp edge of the sample collection pad 240 until a user presses the reservoir into engagement with the sample collection pad. For example, the user can depress the reservoir 250 toward the sample collection pad 240 until the sample collection pad pierces the reservoir.

The fluid can then diffuse onto a lateral flow assay strip retained by receptacle 230. In this aspect, first portion 212 can be at least partially transparent, such that the colorimetric region of the lateral flow assay strip can be observed without the need to move the device to an open position. Optionally, the second portion 214 can be at least partially transparent so that the user can view fluid transfer as disclosed herein.

Optionally, reservoir 250 can also contain an adsorbent material that is saturated (or infused with) with the fluid. One advantage of the adsorbent material is that the fluid will not drain out of the reservoir after the foil is pierced, and will instead transfer to sample collection pad 240. This optional adsorbent material can be useful when the reservoir is unsealed in an orientation where gravity can drain the fluid within the device.

Another exemplary embodiment of a pen-type lateral flow assay device is depicted in FIGS. 4A-5. Referring to FIG. 4A, the lateral flow assay device 300 includes a separable housing 305 having a first housing portion 310 (depicted in FIG. 4A as a female housing portion) that can be securedly coupled to a second housing portion 320 (depicted in FIG. 4A as a male housing portion). First housing portion 310 and second housing portion 320 can in some aspects be threadedly secured to one another to move the housing from the open position to the closed position. The first housing portion 310 and second housing portion 320 can each define a respective interior space, with the interior spaces of the first and second housing portions cooperating to define an enclosed spaced 326 when the two housing portions are secured together.

The enclosed space can include a receptacle 330 configured to receive a lateral flow assay strip 340. For example, the receptacle 330 can define an opening 332 through which the lateral flow assay strip 340 can be received. Lateral flow assay strip 340 can be in fluid communication with, and in some aspects physically connected to, sample collection pad 350. For example, lateral flow assay strip 340 can be configured to physically engage collection pad 350, such that when fluid accumulates on collection pad 350, the fluid will diffuse directly onto lateral flow assay strip 340.

In one aspect, when the housing 305 is in the closed position, the enclosed space 326 defined by the first housing portion 310 and the second housing portion 320 can be divided and sealed by sealing member 360. Sealing member 360 can in general be any fluid resistant barrier, such as an O-ring, gasket, or flange created from the material used for the housing or another suitable material. Although sealing member 360 is shown in FIG. 4A as being part of second housing portion 320, a sealing member can alternatively be present as part of either the first portion 310 or the second portion 320 as long as there is a fluid-resistant/fluid-tight interface between first portion 310 and second portion 320. In further aspects, the sealing member 360 can form a seal between first portion 310 and second portion 320 of housing 305 to fluidly isolate the enclosed space 326 from exterior elements and contamination. In exemplary optional aspects, it is contemplated that engagement between the housing portions and the sealing member 360 can at least partially provide coupling between the first and second housing portions.

Within the interior space defined by first housing portion 310, for example at the top or end of the interior space opposing collection pad 350, there can be a fluid reservoir 370 that is configured to allow fluid transfer from the reservoir to sample collection pad 350 when the housing is in the closed position and when the fluid reservoir is breached. For example, the reservoir 370 can comprise a body 375 and a cap 380 (e.g., a pierceable foil) that is coupled to the body 375 to form a sealed enclosure that defines the fluid reservoir. The cap 380 can be coupled to the body 375 by a heat seal as is known for forming blister packaging. In some aspects, the fluid reservoir 380 can be breached by creating an opening in cap 380, e.g., by piercing cap 380 when the cap comprises a pierceable material such as foil in order to release the fluid to the sample collection pad 350.

In one aspect, for example, the second (male) housing portion 320 can be axially advanced or translated (manually or otherwise) into the first (female) housing portion 310 when the second housing portion is coupled to (e.g., slidingly received within) the first housing portion. When male housing portion 320 is sufficiently axially advanced into the female housing portion 310, sample collection pad 350 can pierce through cap 380, allowing fluid to flow from reservoir 370 onto collection pad 350. Once fluid flows from the reservoir to the collection pad, the fluid can capture a portion of any sample collected on the collection pad and proceed to diffuse onto lateral flow assay strip 340, where the presence or absence of an analyte can be detected visually by observing a colorimetric region of lateral flow assay strip 340 through a transparent region of second housing portion 320. In other words, once the first and second housing portions are secured together after a sample has been collected onto sample collection pad 350, the presence or absence of an analyte can be determined without having to open the housing 305 to the environment, minimizing sample contamination and risks to the user. In some aspects, the male housing portion 320 can be transparent to permit viewing of at least a portion of the lateral flow assay strip 340. In further aspects, female housing portion 310 can be transparent so that the user can view fluid transfer.

In some optional aspects, the male housing portion 320 can be advanced into the female housing portion 310 against a biasing element, such as, for example and without limitation, a spring, which can allow a user to translate the male housing portion relative to the female housing portion while providing resistance to the translation of the male housing portion. When the user releases pressure from the male housing portion, the spring or other biasing element can return the male housing from its translated position (following advancement within the female housing) to its initial position (prior to being axially advanced within the female housing).

Additional, optional details of the embodiment of FIGS. 4A-4B are depicted in FIG. 5. Device 300 in FIG. 5 includes female housing portion 310 configured to receive at least a portion of male housing portion 320. An interior space 322 defined by male housing portion 320 can be configured to receive at least a portion of receptacle 330. Receptacle 330 can receive and secure lateral flow assay strip 340. In one aspect, receptacle 330 can be a silicone assay holder. That is, the receptacle 330 can comprise, or be formed from, silicone. Optionally, lateral flow assay strip 340 can be held in place by receptacle 330 using assay strip brace 335. Sample collection pad 350 (depicted in FIG. 5 as a sample collection nib) can be physically secured to lateral flow assay strip 340 through any suitable mechanism, such as collection pad (or nib) brace 345. The exemplary embodiment of FIG. 5, for example, depicts a portion of sample collection pad 350 that is configured to receive or physically engage with lateral flow assay strip 340, which can permit fluid communication between collection pad 350 and assay strip 340, specifically, to allow for fluid transfer from collection pad 350 to lateral flow assay strip 340. In exemplary aspects, the sample collection pad 350 can comprise a pair of legs 352. The legs 352 can define therebetween a slot 354 that receives therein at least a portion of the lateral flow assay strip 340, and the brace 345 can bias the legs against the lateral flow assay strip. The sample collection pad 350 and the lateral flow assay strip 340 can permit fluid communication therebetween by direct contact.

Receptacle 330 can include sealing member 360, which can be any fluid resistant mechanism capable of preventing unwanted leakage or unwanted transfer of fluid from female housing portion 310 to male housing portion 320 when the device is not being used for analyte detection. Exemplary fluid resistant mechanisms that can serve as the sealing member 360 include, for example and without limitation, an O-ring, gasket, or flange.

An interior space 312 defined by female housing portion 310 can be configured to receive and secure fluid reservoir 370, e.g., within an end portion of the interior space that opposes sample collection pad 350 such that when the housing 305 is in a first closed configuration (FIG. 4B), sample collection pad 350 does not pierce through the fluid reservoir. Once the housing 305 is in the first closed configuration, further axial movement of the male housing portion 320 relative to the female housing portion 310 can cause the sample collection pad 350 to pierce fluid reservoir 370 to thereby communicate the fluid from the fluid reservoir, through the sample collection pad, and to the lateral flow assay strip 340. Fluid reservoir 370 can optionally contain an adsorbent material 385 that can be saturated with the fluid contained in the reservoir. The advantage of such a material is that it can prevent fluid from draining into the interior space defined by female housing portion 310, and instead concentrate the fluid onto sample collection pad 350.

Optionally, reservoir 250 can also contain an adsorbent material that is saturated with the fluid. One advantage of the adsorbent material is that the fluid will not drain out of the reservoir after the foil is pierced, and will instead transfer to sample collection pad 240. This optional adsorbent material can be useful when the reservoir is unsealed in an orientation where gravity can drain the fluid within the device. Fluid reservoir 370 can be sealed with any suitable fitting such as cap 380 depicted in FIG. 5. In one aspect, cap 380 is made of a suitable material (e.g., foil) that can be pierced by sample collection pad 350 when male housing portion 320 is axially advanced into female housing portion 310. Sample collection pad 350 can further be configured (e.g., shaped with a sharp tip) to pierce the fluid reservoir 370.

The device in FIG. 5 works in the same (or generally the same) manner as that described with reference to FIGS. 4A-4B. A sample comprising a suspected analyte can be contacted with sample collection pad 350 (received within male housing portion 320), and then the male housing portion 310 can be coupled to (e.g., slidingly received within) female housing portion 310. Male housing portion 320 can then be axially advanced (manually or otherwise) into female housing portion 310, causing collection pad 350 to pierce through cap 380, allowing fluid-saturated adsorbent material 385 (such as foam) within reservoir 370 to release fluid onto sample collection pad 350, which can then diffuse onto lateral flow assay strip 340. Once the fluid diffuses across lateral flow assay strip 340, the presence or absence of an analyte can be detected when the fluid passes through a colorimetric region of the assay strip, which can be visually observed through a transparent region of male housing portion 320 (without requiring the user to separate the two housing portions).

In some aspects, the devices depicted in FIGS. 1-5 can comprise a lateral flow assay strip in the receptacle configured to receive such a strip. In a further aspect, at least a portion of a first end of the lateral flow assay strip can be in fluid communication with the sample collection pad. In a still further aspect, at least a portion of an opposing second end of the lateral flow assay strip can be in fluid communication with a wicking pad, which can be useful to absorb excess fluid as it travels across the lateral flow assay strip. A colorimetric region can be between the first and second ends of the assay strip. As an altered or unaltered analyte passes the colorimetric region of the assay, the presence or absence of a color can indicate whether or not the analyte is detected.

In some aspects, the devices depicted in FIGS. 1-5 can comprise a suitable fluid in the fluid reservoir. Suitable fluids include buffers capable of maintaining a fluid pH of from 6 to 8.5, e.g., from 6 to 8 or from 6 to 7.4. Similarly, the fluid reservoir can comprise phosphate buffered saline and at least one surfactant. In general, suitable fluids are known in the art and can include any fluid used with lateral flow assay detection methods.

Also disclosed herein is a method of using a disclosed device. The method can comprise contacting the sample collection pad with a sample, e.g., when the device is in the open position and at least a portion of the sample collection pad is exposed. Once sample has been contacted with the collection pad, the method can include releasing a fluid from the fluid reservoir, which can cause the fluid to contact the sample collection pad and diffuse onto the lateral flow assay strip. Once the fluid reaches the colorimetric region of the lateral flow assay strip, a response or absence of a response can be detected, which is indicative of the presence or absence of an analyte in the sample.

As described above, in some aspects, fluid can be released from the fluid reservoir when the housing is moved from the open position to the closed position. In other aspects, fluid can be released from the reservoir manually or by another suitable actuating mechanism. A variety of analytes can be detected using the disclosed device and method, including without limitation fentanyl as well as bio-agents.

### C. Lateral Flow Assay Methods

Also described herein are lateral flow assay methods. In general, the methods can be used with a disclosed device or with another suitable device. The methods allow for lateral flow assay detection of an analyte within a closed environment, such that all consumables needed to conduct a test are contained in a single enclosed environment.

According to some aspects, the method can comprise contacting a sample with a sample collection pad that is in fluid communication with a lateral flow assay strip, causing a fluid to be released onto the sample collection pad (e.g., manually or by another actuation mechanism. Releasing fluid onto the sample collection pad can thereby cause at least a portion of the sample to diffuse onto the lateral flow assay strip. Once sample passes into the colorimetric region of the lateral flow assay strip, a response can be detected that is indicative of the presence or absence of an analyte in the sample.

The fluid released onto the sample collection pad can be any suitable fluid used with lateral flow assay methods. Some examples include a buffer capable of maintaining a pH of from 6 to 8.5, e.g., 6-8, or 6-7.4. For example, the fluid can comprise phosphate-buffered saline and at least one surfactant.

In some aspects, the fluid is released onto the sample collection pad and the sample is diffused onto the lateral flow assay strip within a closed housing, thereby minimizing contamination and potential hazards to the user. According to one aspect, the response from the lateral flow assay strip is detected by observing the lateral flow assay strip through a transparent region of a closed housing, such that the housing does not need to be opened to detect the presence or absence of the analyte.

Those skilled in the art will recognize, or be able to ascertain using no more than routine experimentation, many equivalents to the specific embodiments of the device and methods described herein. Such equivalents are intended to be encompassed by the following claims.

What is claimed is:

1. A device comprising:
a) a housing moveable from an open position to a closed position, wherein the housing in the closed position defines an enclosed space;
b) a receptacle disposed within the enclosed space, wherein the receptacle is configured to receive a lateral flow assay strip;
c) a sample collection pad, wherein at least a portion of the sample collection pad is received within the enclosed space when the housing is in the closed position, and wherein the sample collection pad is positioned to allow fluid transfer from the sample collection pad to the lateral flow assay strip when the lateral flow assay strip is in the receptacle; and
d) a reservoir configured to release fluid to the sample collection pad when the housing is in the closed position.

2. The device of claim 1, wherein the housing comprises at least one transparent region that is positioned relative to at least a portion of the lateral flow assay strip along an axis when the lateral flow assay strip is in the receptacle and when the housing is in the closed position in order to permit viewing of the lateral flow assay strip through the at least one transparent region.

3. The device of claim 1, wherein the housing or the sample collection pad comprises a structure that is configured to pierce the reservoir when the housing is in the closed position, thereby releasing the fluid from the reservoir.

4. The device of claim 1, wherein the housing comprises a first housing portion and a second housing portion, wherein the first and second housing portions cooperate to define the enclosed space.

5. The device of claim 4, wherein the enclosed space comprises a first interior space defined by the first housing portion and a second interior space defined by the second housing portion, and wherein the reservoir is received within the first interior space, and wherein the receptacle configured to receive the lateral flow assay strip is positioned within the second interior space.

6. The device of claim 4, wherein at least a portion of the first housing portion is configured to move axially relative to the second housing portion until the sample collection pad pierces the fluid reservoir.

7. The device of claim 4, wherein the first housing portion is configured to threadedly couple to the second housing portion.

8. The device of claim 1, wherein the reservoir comprises an adsorbent material.

9. The device of claim 1, wherein the reservoir is configured to release the fluid when subject to a user-applied pressure.

10. The device of claim 1, further comprising the lateral flow assay strip in the receptacle, wherein at least a portion of a first end of the lateral flow assay strip is positioned to receive fluid from the sample collection pad.

11. A method of using the device of claim 10, the method comprising:
contacting the sample collection pad with a sample;
releasing a fluid from the fluid reservoir, thereby causing the fluid to contact the sample collection pad and diffuse onto the lateral flow assay strip; and
detecting a response from the lateral flow assay strip that is indicative of the presence or absence of an analyte in the sample.

12. The method of claim 11, wherein releasing the fluid from the fluid reservoir comprises moving the housing to the closed position to thereby release the fluid from the fluid reservoir.

13. The method of claim 11, wherein releasing the fluid from the fluid reservoir comprises releasing the fluid from the fluid reservoir manually.

14. The method of claim 11, wherein the fluid is a buffer capable of maintaining of pH of from 6 to 8.5.

15. The method of claim 11, wherein the fluid comprises phosphate-buffered saline and a surfactant.

16. A method of analyzing a sample, the method comprising:
contacting the sample with a sample collection pad that is in fluid communication with a lateral flow assay strip;
causing a fluid to be released onto the sample collection pad, thereby diffusing at least a portion of the sample onto the lateral flow assay strip; and
detecting a response from the lateral flow assay strip that is indicative of the presence or absence of an analyte in the sample,
wherein the method is carried out in a closed environment.

17. The method of claim 16, wherein the fluid is a buffer capable of maintaining of pH of from 6 to 8.5.

18. The method of claim 16, wherein the fluid comprises phosphate-buffered saline and a surfactant.

19. The method of claim 16, wherein the fluid is released onto the sample collection pad and the sample is diffused onto the lateral flow assay strip within a closed housing.

20. The method of claim 19, wherein the response from the lateral flow assay strip is detected by observing the lateral flow assay strip through a transparent region of the closed housing.
